(19) United States
(12) Patent Application Publication
(10) Pub. No.: US 2008/0096839 A1
(43) Pub. Date: Apr. 24, 2008

(54) Small Interfering Rna and Pharmaceutical Composition for Treatment of Hepatitis B Comprising the Same

(75) Inventors: Meehyein Kim, Seoul (KR); Duckhyang Shin, Kyonggi-do (KR); Soo In Kim, Kyonggi-do (KR); Mahnhoon Park, Kyonggi-do (KR)

Correspondence Address: SUGHRUE MION, PLLC, 2100 PENNSYLVANIA AVENUE, N.W., SUITE 800, WASHINGTON, DC, 20037, US

(73) Assignee: Mogam Biotechnology Research Institute, Yongin-si (KR)

(21) Appl. No.: 11/908,159

(22) PCT Filed: Mar. 9, 2006
(86) PCT No.: PCT/KR06/00837

§ 371 Date: Sep. 10, 2007

Related U.S. Application Data
(60) Provisional application No. 60/660,132, filed on Mar. 9, 2005.

(51) Int. Cl. A61K 31/7105; A61P 31/14; C07H 21/02; C12N 15/63

(52) U.S. Cl. 514/44; 435/320.1; 536/24.5

(57) ABSTRACT

The present invention relates to RNA interference mediated inhibition of Hepatitis B virus (HBV) by short interfering RNA (siRNA) molecules. Specially, siRNAs of the present invention which are double-stranded RNAs concern directing the sequence-specific degradation of viral RNA in mammalian cells. Disclosed is a DNA vector encoding the RNA molecules and synthesized siRNA molecules as well as method of therapeutic treatment for inhibition of HBV gene expression and viral replication by the administration of RNA molecules of the present invention.

The instant patent application claims priority to U.S. Application Ser. No. 60/660,132 filed on Mar. 9, 2005. The instant application claims the benefit of the listed application, which is hereby incorporated by reference herein in its entirety.

### TECHNICAL FIELD

The present invention relates to a small interfering RNA specific for Hepatitis B virus X gene and the pharmaceutical use thereof.

### BACKGROUND ART

It is estimated that over 300 million people worldwide are chronically infected with Hepatitis B virus (HBV). Patients with HBV-associated liver failure may develop liver cirrhosis or hepatocellular carcinoma. One of the major anti-HBV therapies is treatment of interferon-alpha or lamivudine, or combination therapy with both of them. However, interferon-alpha as an anti-viral drug shows shortcomings, such as the low efficacy, side effects and high costs. Lamivudine, a nucleoside analogue, is a very potent and specific inhibitor to HBV reverse transcriptase. Nonetheless, it causes the viral genomic mutation resistant to the drug and a reactivation of viral replication by cessation of the treatment in patients. Only about 20% of the HBV patients response to combination therapy with interferon-alpha and lamivudine.

HBV is a small enveloped DNA virus and belongs to hepadnaviridae. Human liver is the primary target organ of HBV. HBV infection usually leads to severe liver failure, such as chronic hepatitis, cirrhosis or hepatocellular carcinoma. HBV genome is a partial double-stranded circular DNA with length of 3.2 kb that contains four open reading frames, called S, C, P and X. Transcription of genomic DNA produces four different viral RNAs that are of size 3.5 (pregenomic RNA), 2.4, 2.1, and 0.7 kb (message RNAs) See FIG. 1. (Ganem and Varmus, Annu. Rev. Biochem., 1987, 56, 651). The pregenomic RNA plays critical roles for not only translation of viral proteins but also reverse-transcription of viral DNA by polymerase protein. The core protein packages partial circular DNA and polymerase protein followed by the nucleocapsid assembly. And then the nucleocapsid particle interacts with viral envelop proteins to form mature infectious virions that are secreted out of the cell at the last step of viral life cycle.

HBV X (HBx) gene is the smallest, with length of 465 nucleotides and encodes HBx protein that is 154 amino acids long with a molecular weight of 17 kDa (Fujiyama et al., Nucleic Acids Res., 1983, 11, 4601). It is a pleiotropic transactivator to stimulate not only the HBV promoters and enhancers, but also a wide range of other viral promoters via protein-protein interaction (Nakatake et al., Virology, 1993, 195, 305; Spandau and Lee, J. Virol., 1988, 62, 427). Moreover, the HBx protein is a critical element inducing cellular transformation and liver tumors either through interaction with cellular transcription factors or through a signal transduction pathway (Kekule et al., Nature, 1993, 361, 742). As the HBx protein is implicated in HBV-mediated HCC and its coding region is contained in all of the four HBV mRNAs and highly conserved in a wide range of HBV subtypes, HBx gene must be an ideal target to design and develop the anti-HBV siRNAs.

The viral life cycle can be initiated and propagated artificially by transfection of the HBV genomic plasmid (of adr subtype of gene-bank access no. M38636), pcDNA-HBV1.3, to introduce the viral replication system. See FIG. 2. The pcDNA-HBV1.3 clone was developed by modification of the previously reported protocol (Guidotti et al., J. Virol., 1995, 69, 6158). Transfection of the HBV genomic plasmid leads to the expression of viral RNAs and proteins in vitro. It can be also applied to construct an in vivo mouse model system, in which the complete immune responses and viral replication and assembly of mature viral particles are accompanied by hydrodynamic injection of a naked plasmid DNA bearing the HBV genome into tail veins of mice. This is a powerful tool to mimic and induce the viral replication cycle experimentally and to monitor the efficiency of antiviral drugs by detection of viral proteins or observation of viral nucleic acids. For example, co-injection of the HBV complete plasmid together with siRNA or its expression vector caused a significant inhibition in the level of viral antigens, transcripts and replicative DNA in the livers and sera (Giladi, Molecular Therapy, 2003, 8, 769; McCaffrey, Nat. Biotechnol., 2003, 21: 639).

In the meantime, RNA interference (RNAi) is evolutionally conserved process in which (endogenous and exogenous) gene expression is suppressed by introduction of double-stranded RNA (dsRNA) in all eukaryotes. RNAi is initiated by an RNase III-like endonuclease, called Dicer, which promotes consecutive cleavage of long dsRNAs into 21-23 nt short interfering RNAs (siRNAs) (Bernstein et al., Nature, 2001, 409, 363). siRNAs are incorporated into an RNA-induced silencing complex (RISC), which unwinds the siRNA in the presence of ATP (Hammond, et al., Nature, 2000, 404, 293). The antisense RNAs incorporated into RISC recognize the homologous RNAs and direct their degradation in the cellular cytoplasmic region.

The dsRNA over 30 nt in length induces a nonspecific interferon (IFN) response that activates protein kinase R (PKR) and RNase L (Balachandran et al., Immunity, 2000, 13, 129). The induction of PKR and RNase L activity finally leads to mRNA degradation and represses mRNA translation, nonspecifically, in mammal cells. However, siRNAs are short enough to bypass the interferon pathway and direct gene silencing with sequence specificity (Elbashir et al., Nature, 2001, 411, 494). Generation of siRNA is expected to protect against genetic invasion caused by transposons, transgenes and viruses, which partially or completely harbor long dsRNA elements (Plasterk, Science, 2002, 296, 1263; Zamore, Science, 2002, 296, 1265; Hannon, Nature, 2002, 418, 244).

Many trials have been performed to select siRNAs to inhibit the replication of pathogenic RNA viruses, such as human immunodeficiency virus (HIV), hepatitis C virus (HCV), poliovirus, and so on (Novina et al., Nat. Med., 2002, 8, 681; Wilson et al., Proc. Natl. Acad. Sci. USA, 2003, 100, 2783; Getlin et al., Nature, 2002, 418, 430).

However, there is no known effective anti-viral inhibitor including siRNA molecules to inhibit the replication of hepatitis B viruses up to date.

Thus, it is required to develop urgently an anti-viral inhibitor to treat HBV infected patients.

As HBV pregenomic RNA is a key intermediate to maintain viral DNA replication via reverse transcription in the virus life cycle, it is a reasonable candidate for RNAi. Consequently, the present inventors invented the present invention by paying attention to an applicability of siRNA specific for the HBV pregenomic RNA and finding that a series of siRNAs specific for Hepatitis B virus X gene could inhibit of viral replication and gene expression.

### DISCLOSURE OF INVENTION

### Technical Problem

The object of the present invention is to provide a pharmaceutical agent effective in treating hepatitis B.

### Technical Solution

In order to achieve the object, the present invention provides a small interfering RNA molecule (siRNA) specific for Hepatitis B virus X gene. This invention is based on the discovery siRNA molecules by targeting HBV X gene, which induces degradation of HBV pregenomic RNA and message RNAs, and finally inhibits the expression of viral proteins and the viral replication.

### BRIEF DESCRIPTION OF THE DRAWINGS

FIG. 1 is a schematic diagram illustrating the location of siRNA target sites specific for HBV X gene. Downward arrows indicate the target sites within the HBV RNA transcripts. The ORFs are drawn below aligned with the HBV mRNAs:

P: polymerase; C: HBcAg;

S1: large pre-surface antigen; S2: middle pre-surface antigen;

S: HBsAg; and X: X protein.

FIG. 2 is a schematic presentation of HBV1.3:

Enh: enhancer; X: X gene; C: core gene;

S1: preS1 gene; S2: preS2 gene; and S: S gene.

FIG. 3 is a schematic diagram illustrating a pRNAiDu siRNA expression cassette. To construct the pRNAiDu vector, the human U6 and human Hi promoter sequences were cloned in the opposite direction. Appropriate mutations were induced to define termination signals for siRNA transcription by the RNA polymerase III or facilitate ligation of siRNA-encoding oligomers.

FIG. 4 is a graph showing relative levels of HBsAg in culture media of siRNA expression vector-transfected cell. The HBsAg levels were measured at day 1, 2 and 3, following standardization of the transfection efficiency via FLuc assay as an internal control.

FIG. 5 is a graph showing dose-dependant kinetics of inhibition of HBsAg expression by synthetic siRNA. Huh-7 cells (4×10⁵) were transfected with 0.5□ of pcDNA-HBV1.3 and the indicated amount of the synthetic HBx-1 siRNA or control siRNA, and assayed for the amount of HBsAg secreted into the media at day 1, 2, and 3 after transfection. The amount HBsAg by HBx-1 siRNA are shown as percentages of the amounts secreted by control siRNA-transfected cells.

FIG. 6 is a series of photographs showing detection of the synthetic siRNA in the mouse liver. The synthetic double-stranded siRNA labeled with fluorescein was delivered into mice by hydrodynamic tail vein injection. After 20 hour postinjection, liver was dissected via cryosection and exposed on the fluorescence microscopy. Liver cells with fluorescence labeled siRNAs are indicated with arrows.

FIG. 7 is a graph showing serum HBsAg levels in synthetic siRNA-received mice. HBsAg levels in C57BL/6 mouse sera were measured at day 2 after injection with pcDNA-HBV1.3 and 0.5 nmol synthetic siRNA of HBx-1 or control.

FIG. 8 is a graph showing dose-dependent inhibition of HBsAg expression in mice. Mice were injected with 10□ of pcDNA-HBV1.3 DNA separately, or together with increasing amounts of synthetic siRNA of HBx-1 or control, and monitored for the levels of HBsAg after 2 days.

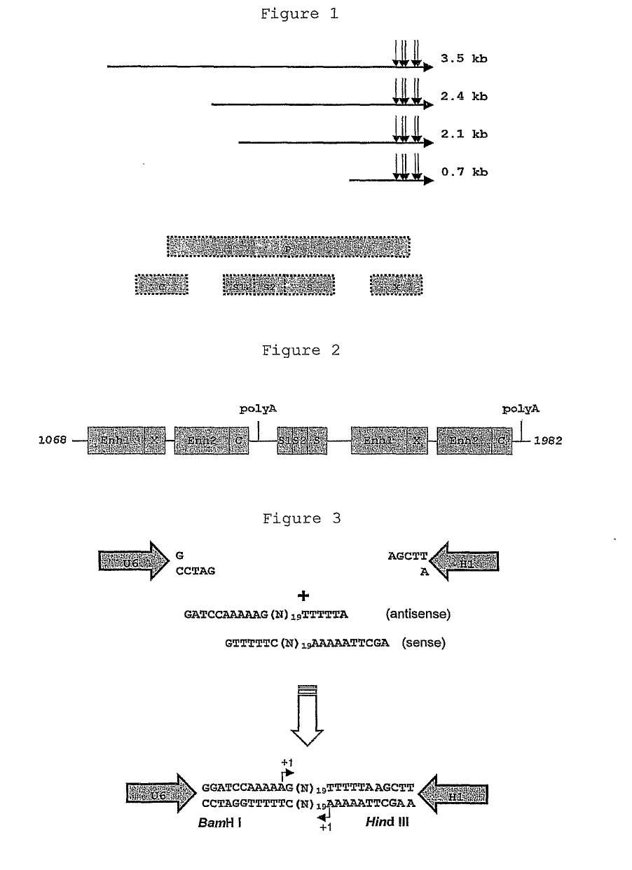

### BEST MODE FOR CARRYING OUT THE INVENTION

This invention is based on the discovery siRNA molecules by targeting HBV X gene, which induces degradation of HBV pregenomic RNA and message RNAs, and finally inhibits the expression of viral proteins and the viral replication.

In some embodiments, the siRNA is obtained by hybridization of the two complementary synthetic RNAs or transfection of a vector encoding the RNA in the cell. For efficient inhibition of the viral replication, siRNA sequences for the target segments on the HBV X gene were selected from the group of following SEQ. ID. NOs: 1-5, a complement thereof, or a portion thereof:

| | | |
|---|---|---|
| HBx-1: | 5′-GAGGACUCUUGGACUCUCA-3′; | (SEQ. ID. NO:1) |
| HBx-2: | 5′-UGUCAACGUCCGACCUUGA-3′; | (SEQ. ID. NO: 2) |
| HBx-3: | 5′-CGUCCGACCUUGAGGCAUA-3′; | (SEQ. ID. NO: 3) |
| HBx-4: | 5′-UGAUCUUUGUACUAGGAGG-3′; | (SEQ. ID. NO: 4) |
| and | | |
| HBx-5: | 5′-AUUGGUCUGUUCACCAGCA-3′. | (SEQ. ID. NO: 5) |

In an embodiment, the present invention provides an isolated nucleic acid molecule comprising a nucleotide sequence selected from the group of SEQ. ID. NOs: 1 to 5, or a complement thereof, or a portion thereof.

In a preferred embodiment, the isolated nucleic acid molecule is a single stranded nucleic acid molecule.

In another preferred embodiment, the isolated nucleic acid molecule further comprises a complementary strand of said isolated nucleic acid molecule, which can hybridize with the same.

In a preferred embodiment, the isolated nucleic acid molecule is a short interfering RNA (siRNA).

In a more preferred embodiment, the complementary strands of the siRNA are covalently connected via a linker molecule.

In another preferred embodiment, the linker molecule is a polynucleotide linker or a non-nucleotide linker.

In further preferred embodiment, the nucleic acid molecule binds to a HBV X gene.

The present invention provides a method for treatment of an infectious disease related to HBV, comprising administrating to the subject pharmaceutically effective amount of a double-stranded siRNA molecule comprising a nucleotide sequence selected from the group of SEQ. ID. NOs: 1 to 5, or a complement thereof, or a portion thereof.

Also, the present invention provides a DNA vector comprising a DNA sequence corresponding a nucleotide sequence selected from a group of SEQ. ID. NOs: 1 to 5, or a complement thereof, or a portion thereof.

In a preferred embodiment, the DNA vector of the present invention is suitable for expression of siRNA.

In addition, the present invention provides a pharmaceutical composition comprising the isolated nucleic acid molecule described above or the DNA vector and pharmaceutically acceptable carriers or excipients, for treating, preventing or diagnosing hepatitis B, liver cirrhosis or liver cancer.

To increase the stability of siRNA or the specific interaction between viral target RNA region and siRNA fragment, the 3′ends of both of the two strands of siRNA were extended with dTdT or UU, by chemical synthesis. In some embodiments, synthetic siRNA can be modified by chemical derivatives or tagging molecules for acquiring its physiological stability and chasing its distribution in the cell.

In some preferred embodiments, each strand of double-stranded siRNA is expressed from the two separated promoters, in opposite or in parallel, and hybridizes with its complement in the living cell. Alternatively, shRNA can be transcribed from a single promoter independently and processed into double-stranded siRNA by cellular Dicer, following induction of degradation of target RNA. A vector expressing siRNA contains not only promoter(s) for initiation of transcription but also enhancer, transcription termination signal, or other expression regulatory sequences. The vector can be delivered into the cellular nucleus as a naked plasmid DNA, a complex with transfection reagent or target-delivery material, or as a form of recombinant viral vector. The construction of the vector is determined by specific situations, such as the cell state or type to be transfected, the time and level of siRNA expression, and so on.

The present invention demonstrates a DNA vector that transcribes double-stranded siRNA from the two convergent promoters. The vector, partially or completely, inhibits HBV gene expression and viral replication in the cell. RNA interference effect is dependant on the detection time and transfected DNA dose and causes over 90% of inhibition of viral RNA accumulation or protein expression. Specially, the siRNA expression cassette, separated from the vector by restriction endonucleases, is an efficient element inducing the RNAi effect.

The invention also demonstrates the RNAi activity induced by synthetic siRNA in which 3′ end of each strand RNA in extended with dTdT for its stability. The synthetic RNA efficiently inhibits accumulation of viral RNA and gene expression by 98% in the cell and by 97% in the HBV mouse model, respectively. In the mouse, it is observed that the fluorescein labeled siRNA is delivered into the liver tissue by hydrodynamic injection. It will be a new therapeutic approach for treating a hepatitis viral carrier, infected by HBV, by administration to a subject in need thereof synthetic siRNA or vector.

The present invention demonstrates a therapeutic application of synthetic siRNA or vector encoding double-stranded siRNA and the combination therapy containing siRNA to inhibit HBV replication in its carriers.

### MODE FOR THE INVENTION

This invention relates to siRNA molecules specific for Hepatitis B virus X gene and their application for the clinical treatment to hepatitis B virus (HBV) chronic carrier to inhibit viral replication and gene expression.

An siRNA of the present invention can be synthesized chemically or enzymatically (Caruthers et al., Methods in Enzymology, 1992, 211, 3; Wincott et al., 1995, Nucleic Acids Res., 23, 2677; Brennan et al., Biotechnol. Bioeng., 1998, 61, 33).

An siRNA or vector of this invention can be delivered to target cells using transfection carriers, such as liposomes, hydrogels, bioadhesive microspheres and the like (Akhtar et al., Trends Cell Bio., 1992, 2, 139).

A pharmaceutical composition contains an siRNA or vector of this invention with an organ targeting material and a pharmaceutically acceptable carrier for treating an infection with HBV. The dose of pharmaceutical composition can be determined, therapeutically, by a specific situation, such as the route of administration, the nature of the formulation, the phase of liver failure, the subject's size, weight, or distribution range, and the age and sex of patient.

Practical and presently preferred embodiments of the present invention are illustrative as shown in the following Examples.

However, it will be appreciated that those skilled in the art, on consideration of this disclosure, may make modifications and improvements within the spirit and scope of the present invention.

### EXAMPLE 1

### Constructing of a siRNA Expression Vector

In mammalian cells, previously siRNA vector has been designed to transcribe short hairpin RNAs (shRNAs) from an RNA polymerase III promoter (such as U6, Hi, or tRNA promoter) or a polymerase II promoter with a poly(A) signal sequence (Brummelkamp et al., Cancer Cell, 2002, 2, 243; Tushcl, Nat. Biotechnol., 2002, 20, 446; Xia et al., Nat. Biotechnol., 2002, 20, 1006). However, shRNA vectors show multiple drawbacks. Their non-natural secondary structure induces that it is hard to synthesize them in bacteria and to sequence, and DNA oligomers to generate them can be costly in the case of high through-put screening. Moreover, it is no facile to generate an siRNA expression cassette containing a promoter to a termination signal without additional tag-sequences for constructing diverse siRNA library. To circumvent these limitations of shRNA expression vectors, we constructed a vector for direct expression of siRNA, which is transcribed from convergent opposing promoters, and named it pRNAiDu (Kaykas and Moon, BMC Cell Biology, 2004, 5, 16; Zheng et al., Proc Natl. Acad. Sci. USA, 2004, 101, 135). See FIG. 3.

Both the human U6 and Hi promoters were modified to contain polymerase III termination sequences of five thymidine nucleotides at the −5 to −1 position and a Bam H I site and a Hind III site at each −12 to −6 position, respectively. As the U6 promoter prefers a purine nucleotide for transcription initiation, guanidine is inserted at the +1 position downstream of the U6 promoter. To minimize an artificial effect of induced this additional nucleotide and guarantee a consecutive hybridization between antisense siRNA and target RNA in the RNAi process, it was devised that the U6 promoter takes a charge of transcription for the antisense RNA, which directs RISC to cleave the homologous mRNA. To create the siRNA expression plasmids, pairs of 36-base oligonucleotides were annealed and ligated into pRNAiDu digested with BamH I and Hind III. Specially, in the pRNAiDu vector, the fusion gene of enhanced green fluorescent protein (EGFP) and firefly luciferase (FLuc), EGFP-FLuc, is contained under the SV40 promoter. Experimentally, this is useful to visualize and quantitatively monitor the transfection efficiency, and to standardize the RNAi activity via detection of fluorescence or luminescence.

### EXAMPLE 2

### Inhibition of HBsAg Expression by HBV siRNAs In Vitro

The Huh-7 cells were seeded at a subconfluent density of 4×10⁵ cells in 6 well culture plates. One day after, the cells were transfected with 0.5 μg of pcDNA-HBV1.3 and 1.5 μg of pRNAiDu, as a control vector, or a siRNA vector, using Lipofectamine 2000 (Invitrogen, USA) following the user guideline. At 1, 2 and 3 days after transfection, media were collected for quantitative detection of the level of HBsAg, and the cells were harvested for standardization of the transfection efficiency using firefly luciferase assay kit (Promega, USA). Experiments were performed in triplicate.

The levels of HBsAg in 100 μl of the media of the transfected cells were measured using HBsAg enzyme immunoassay kit (DiaSorin, Italy).

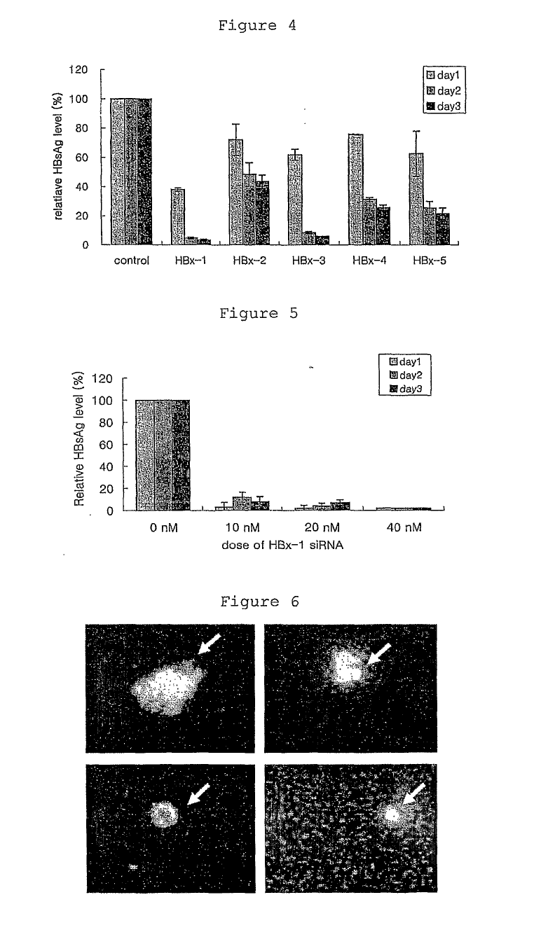

To investigate the anti-viral activity of the HBV siRNAs, the levels of the secreted HBsAg in the culture media were quantified at 1, 2 and 3 days after transfection. See FIG. 4. The transfection efficiency in each experiment set was corrected by measuring the amount of FLuc protein in the cell lysates treated with the siRNA expression vectors. Compared with the control siRNA vector, HBsAg expression by HBV siRNA vectors was reduced by 80% in average in the cells at day 3 after transfection. Among all the siRNA expression vectors, pRNAiDuHBx-1 and pRNAiDuHBx-3 exhibited the most dramatic inhibition, as HBsAg were reduced 97% and 94% at day 3 post-transfection, respectively. It means that the strong siRNAs targeting HBx gene efficiently inhibit not only viral replication but also expression of other HBV genes by simultaneous degradation of all kinds of viral pregenomic and mRNAs containing homologous target X gene.

To examine whether the siRNA expression cassette from the U6 promoter to the Hi promoter is enough to induce the siRNA-medicated RNA interference, the cassette was separated from the siRNA expression vector by digestion with restriction endonuclease. The linearized siRNA vectors were co-delivered with the HBV complete genome plasmid into Huh-7 cells. The results indicate that the linearized siRNA cassette, as well as the circular siRNA expression plasmid, is also able to induce the RNAi effect with decrease of the HBsAg level by about 90% in the media. See Table 1. This suggests that the siRNA expression cassette with two RNA polymerase III promoters, convergently opposing, is a useful tool to develop the PCR product-based anti-HBV gene therapeutics.

TABLE 1
RNAi effect of the linearized siRNA expression cassette.

Relative amount of HBsAg (%)

| | control | HBx-1 |
|---|---|---|
| circular plasmid | 100 | 8 ± 0.7 |
| linearized plasmid | 100 | 6 ± 0.5 |
| (EcoR I) | | |

To confirm further the inhibitory effect of siRNA on HBV gene expression, we prepared synthetic siRNAs of control siRNA and HBx-1 siRNA. Then we conducted dose-response analysis by co-delivery with 0.5 μg of pcDNA-HBV1.3 and increasing amounts of synthetic siRNA into the Huh-7 cells and by monitoring the level of HBsAg secreted into the media at day 1, 2 and 3 posttransfection. See FIG. 5. The results reveal that at least 10 nM of synthetic HBx-1 siRNA is sufficient for inducing strong inhibitory effect of HBV gene expression at day 1 (over 90%), comparing with control siRNA. Moreover, in the case of exposure of the HBV replication complete vector into the 40 nM synthetic HBx-1 siRNA, HBsAg protein was totally exhausted down to undetectable level. This definite inhibitory effect appears to last for 3 days. These results in vitro suggest that HBx-1 siRNA must be a specific and strong inhibitor and an ideal candidate for silencing of viral gene expression via RNA interference process.

### EXAMPLE 3

### Reduction of Viral Transcripts by HBV siRNAs In Vitro

Total RNA was extracted from Huh-7 cells (about 10⁶) delivered with pcDNA-HBV1.3 and either control siRNA vector or HBV-specific siRNA vector, at day 2 posttransfection, using Trizol LS reagent (Invitrogen, USA) according to the manufacturer's instruction. The isolated total RNA was digested with RNase-free DNase (Promega, USA). Finally, absolute amount of RNA was determined by measuring UV-absorbance at 260 nm/280 nm using UV spectrophotometer.

Antiviral activity was assessed by means of a quantitative real time RT-PCR (Sequence Detection System 5700, Applied Biosystems, USA). The real time RT-PCR was performed with 500 ng of total RNAs isolated from the transfectants in a reaction volume of 50 μl using the TaqMan One-Step RT-PCR Master Mix Reagents (Applied Biosystems, USA). The primer and probe sequences, specific for HBV X gene, include

5′-TCCCCGTCTGTGCCTTCTC-3′
(forward primer, SEQ. ID. NO: 6),
5′-GTGGTCTCCATGCGACGTG-3′
(reverse primer, SEQ. ID. NO: 7) and
5′(fluorescein)-CCGGACCGTGTGCACTTCGCTT(TAMRA)-3′.
(probe, SEQ. ID. NO: 8)

The total RNA amount was corrected, definitely, by carrying out real time RT-PCR targeting human β-Actin gene as an internal control, in parallel. The primer and probe sequences for β-Actin gene include 5′-GCGCGGCTACAGCTTCA-3′ (forward primer, SEQ. ID. NO: 9), 5′-TCTCGTTAATGTCACGCACGAT-3′ (reverse primer, SEQ. ID. NO: 10) and 5′-(fluorescein)CACCACGGCCGAGCGGGA(TAMRA)-3′(probe, SEQ. ID. NO: 11). All experiments were performed in triplicate.

To determine whether HBV siRNA vector can reduce the viral RNA level in vitro, we monitored the RNAi activity induced by HBx-specific siRNA vectors using quantitative realtime RT-PCR. The relative amount of viral RNA transcripts was presented as percentages of the control siRNA vector. See Table 2. Compared with a control vector, pRNAiDu, significant reduction of the viral transcripts was detected when siRNA vector targeting specific HBx RNA were used. Specially, much more dramatic reduction of viral RNA was detected by 70% and 60% in the total RNA prepared from cells transfected with pRNAiDuHBx-1 and pRNAiDuHBx-3, respectively, on day 2 posttransfection. These results demonstrate that RNAi can efficiently induce viral RNA degradation and inhibit HBV replication in cultured Huh-7 cells.

TABLE 2
Quantitative measurements (by realtime RT-PCR)
of HBV transcripts in the Huh-7 cells co-transfected
with HBV DNA and siRNA expression vector.

| siRNA | Relative HBx RNA amount (%) |
|---|---|
| control | 100 |
| HBx-1 | 30 ± 0.5 |
| HBx-2 | 46 ± 3.0 |
| HBx-3 | 39 ± 7.6 |
| HBx-4 | 53 ± 9.0 |
| HBx-5 | 51 ± 7.6 |

### EXAMPLE 4

### Inhibition of HBsAg Expression by Synthetic HBx siRNA In Vivo

We performed in vivo experiments with female C57BL16 mice weighing between 18 to 20 g (Orient, Korea). The complete HBV DNA, pcDNA-HBV1.3, and siRNAs were delivered into mice using the hydrodynamic injection method, by which 10 μg of pcDNA-HBV1.3 and 0.5 nmole siRNA dissolved in RNase-free 0.85% NaCl were injected into the mice tail vein (Zhan et al., Hum. Gene Ther., 1999, 10, 1735; Lin et al., Gene Ther., 1999, 6, 1258). In the dose-response assay, mice were injected with 10 μg of pcDNA-HBV1.3 together with increasing amounts of control siRNA or HBx-1 siRNA. The mouse serum was separated by eye-bleeding and assayed for HBsAg level at day 1, 2 and 3 after hydrodynamic injection.

To visualize that synthetic RNA can reach the target organ, we prepared the synthetic double-stranded RNA with 21 nucleotides in length labeled with fluorescein at the 3′ end of sense strand of RNA and injected 1 nmole RNA into the mice tail vein. At 20 h after injection, mice were sacrificed, and the livers were separated and dissected into pieces via cryosection.

By exposure of the pieces of liver section on the fluorescence microscopy, spots with fluorescence were detected after 20 h postinjection. See FIG. 6. It shows that some portion of the synthetic RNA can be delivered to the target organ by escaping the RNase attacks which abundantly distribute everywhere in the serum and the tissue of the mouse. It appears promising that the hydrodynamic injection methods must be a compatible tool to observe the synthetic siRNA-mediated RNAi efficacy in the mouse model.

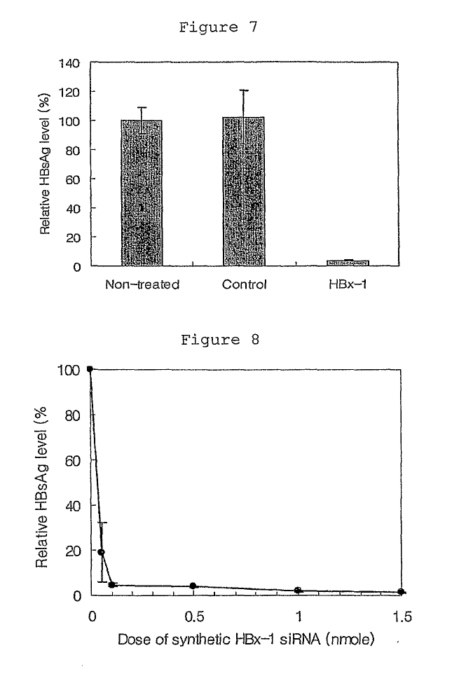

We selected an siRNA with the strongest in vitro inhibition effect on HBV gene expression for confirming its interference effect in the mouse model. By the hydrodynamic injection method, mice were received 10 μg of pcDNA-HBV1.3 plasmid separately, or together with 0.5 nmole of synthetic siRNA of control siRNA or HBx-1 siRNA. After 2 days, we separated serum samples and assessed theirs HBsAg level by performing ELISA assay. See FIG. 7. As expected, the negative control siRNA duplex did not cause reduction of the HBsAg level expressed from the HBV replication competent vector in the mouse. In accordance with the in vitro cell culture experiments, synthetic HBx-1 siRNA induced the prominent inhibition of HBsAg expression by 96% in the sera.

To investigate the dose-dependant response of siRNA for inhibition of viral gene expression, we delivered 10 μg of pcDNA-HBV1.3 plasmid together with 0.05, 0.1, 0.5, 1 or 1.5 nmole of control or HBx-1 siRNA into mice and monitored the level of HBsAg in the serum at day 2 after the hydrodynamic tail vein injection. See FIG. 8. With as little amount of 0.05 nmole HBx-1 siRNA comparing with control siRNA, the HBsAg level was efficiently inhibited by 78%. Furthermore, dose of 0.1 nmole of the HBx specific siRNA was enough amounts for inducing the saturated inhibition effect for HBV gene expression.

To investigate the kinetic inhibitory effect, the sera of mice injected with pcDNA-HBV1.3 and synthetic siRNA was harvested at different time intervals of day 1, 2 and 3 after injection for measuring the HBsAg level. See Table 3. Results of a kinetic study displayed that the HBV gene expression in variable concentrations (0.05-1.5 nmole) of the synthetic RNA reached to undetectable range after day 2. The relative HBsAg levels induced by HBx-1 siRNA were presented as percentages of control siRNA. All experiments were performed in triplicate. In the ELISA assay, the saturated inhibition effect lasted for at least 3 days. This observation suggests that HBx-1 siRNA significantly and efficiently inhibits the viral replication via degradation of sequence specific viral RNAs and inhibition of the gene expression.

TABLE 3
Kinetics of RNAi effect by the HBx-1 siRNA in mice.

| | Relative levels of HBsAg (%) | | |
|---|---|---|---|
| HBx-1 siRNA (nmole) | Day 1 | Day 2 | Day 3 |
| 0.05 | 21.6 ± 6.1 | 19.2 ± 13.2 | 27.3 ± 14.2 |
| 0.10 | 13.3 ± 4.0 | 4.7 ± 0.8 | 5.1 ± 1.3 |
| 0.50 | 9.5 ± 2.5 | 3.8 ± 0.4 | 5.6 ± 2.1 |
| 1.00 | 6.8 ± 0.6 | 1.8 ± 0.4 | 3.7 ± 0.7 |
| 1.50 | 4.4 ± 1.5 | 1.4 ± 0.2 | 3.2 ± 2.2 |

### INDUSTRIAL APPLICABILITY

The present invention relates to a siRNA specific for HBV X gene and a pharmaceutical use thereof. The siRNA of the present invention can be effectively used for treating diseases resulting from infection of hepatitis B virus, since the siRNA induces degradation of HBV pregenomic RNA and message RNAs, and finally inhibits the expression of viral proteins and the viral replication.

### SEQUENCE LISTING

SEQ. ID. NOs: 1˜5 are the nucleotide sequences of the siRNA molecules of the present invention.

SEQ. ID. NO: 6 and SEQ. ID. NO: 7 are primers for real time RT-PCR to detect HBV X gene.

SEQ. ID. NO: 8 is a probe for real time RT-PCR to detect HBV X gene.

SEQ. ID. NO: 9 and SEQ. ID. NO: 10 are primers for real time RT-PCR to detect β-actin gene.

SEQ. ID. NO: 11 is a probe for real time RT-PCR to detect M-actin gene.

1. An isolated nucleic acid molecule comprising a nucleotide sequence selected from the group of SEQ ID NOs: 1 to 5, or a complement thereof, or a portion thereof.

2. The isolated nucleic acid molecule according to claim 1, wherein the nucleotide sequence is SEQ ID NO: 1 or 3.

3. The isolated nucleic acid molecule according to claims 1 or 2, wherein the nucleic acid molecule is a single stranded nucleic acid molecule.

4. The isolated nucleic acid molecule according to any one of claims 1 to 3, further comprising a complementary strand thereof.

5. The isolated nucleic acid molecule according to claim 4, wherein the nucleic acid molecule is a short interfering RNA (siRNA).

6. The isolated nucleic acid molecule according to claims 5, wherein the complementary strands of the siRNA are covalently connected via a linker molecule.

7. The isolated nucleic acid molecule according to claim 5, wherein the linker molecule is a polynucleotide linker or a non-nucleotide linker.

8. The isolated nucleic acid molecule according to claims 1 or 2, wherein the isolated nucleic acid molecule binds to the HBV X gene.

9. A method for treatment of an infectious disease related to HBV, comprising administrating to a subject a pharmaceutically effective amount of double-stranded siRNA molecules comprising the isolated nucleic acid molecule according to any one of claims 1 to 8.

10. A DNA vector comprising a DNA sequence corresponding a nucleotide sequence selected from the group of SEQ ID NOs: 1 to 5, or a complement sequence thereof, or a portion thereof.

11. The DNA vector according to claim 10, wherein the vector is suitable for siRNA expression.

12. A pharmaceutical composition comprising the isolated nucleic acid molecule according to any one of claims 1 to 8 or the DNA vector according to claim 10 or claim 11 and pharmaceutically acceptable carriers or excipients, for treating, preventing or diagnosing Hepatitis B, liver cirrhosis or liver cancer.


SEQUENCE LISTING (11 sequences)

SEQ ID NO: 1 - RNA, 19 nt, Artificial Sequence
  siRNA sequences of the segments on the HBV X gene 1
gaggacucuuggacucuca

SEQ ID NO: 2 - RNA, 19 nt, Artificial Sequence
  siRNA sequences of the segments on the HBV X gene 2
ugucaacguccgaccuuga

SEQ ID NO: 3 - RNA, 19 nt, Artificial Sequence
  siRNA sequences of the segments on the HBV X gene 3
cguccgaccuugaggcaua

SEQ ID NO: 4 - RNA, 19 nt, Artificial Sequence
  siRNA sequences of the segments on the HBV X gene 4
ugaucuuuguacuaggagg

SEQ ID NO: 5 - RNA, 19 nt, Artificial Sequence
  siRNA sequences of the segments on the HBV X gene 5
auuggucuguucaccagca

SEQ ID NO: 6 - DNA, 19 bp, Artificial Sequence
  HBx forward primer for real time RT-PCR
tccccgtctgtgccttctc

SEQ ID NO: 7 - DNA, 19 bp, Artificial Sequence
  HBx reverse primer for real time RT-PCR
gtggtctccatgcgacgtg

SEQ ID NO: 8 - DNA, 22 bp, Artificial Sequence
  HBx probe for real time RT-PCR
ccggaccgtgtgcacttcgctt

SEQ ID NO: 9 - DNA, 17 bp, Artificial Sequence
  b-actin forward primer for real time RT-PCR
gcgcggctacagcttca

SEQ ID NO: 10 - DNA, 22 bp, Artificial Sequence
  b-actin reverse primer for real time RT-PCR
tctcgttaatgtcacgcacgat

SEQ ID NO: 11 - DNA, 18 bp, Artificial Sequence
  b-actin probe for real time RT-PCR
caccacggccgagcggga